(19) United States
(12) Patent Application Publication
(10) Pub. No.: US 2013/0171199 A1
(43) Pub. Date: Jul. 4, 2013

(54) CONTROLLED RELEASE PHARMACEUTICAL COMPOSITION

(71) Applicants: Sanjay Boldhane, Mumbai (IN); Shripad Jathar, Mumbai (IN); Maneesh Nerurkar, Mumbai (IN)

(72) Inventors: Sanjay Boldhane, Mumbai (IN); Shripad Jathar, Mumbai (IN); Maneesh Nerurkar, Mumbai (IN)

(73) Assignee: Abbott Healthcare Private Limited, Mumbai (IN)

(21) Appl. No.: 13/515,482

(22) PCT Filed: Dec. 20, 2010
(86) PCT No.: PCT/IN10/00829

§ 371 Date: Jun. 12, 2012

(30) Foreign Application Priority Data
Dec. 22, 2009 (IN) 2955/MUM/2009

(51) Int. Cl. A61K 9/50

(52) U.S. Cl. CPC A61K 9/50
USPC 424/400; 514/397; 514/629; 514/226.5; 514/652; 514/651; 514/603

(57) ABSTRACT

An oral controlled release pharmaceutical composition comprising a core and a polymer dispersion and its preparation method are disclosed. The core is selected from the group consisting of ‘the drug coated core’ and ‘the drug loaded core’. The drug coated core comprises an inert excipient based sphere and a coat of drug composition. The drug loaded core comprises at least a drug, a binder and at least one pharmaceutically acceptable excipient. The polymer dispersion used to coat the core comprises at least one controlled release polymer and at least one pharmaceutically acceptable excipient. The oral controlled release pharmaceutical composition further comprises an in-situ gelling system comprising at least one gelling polymer.

### FIELD OF THE INVENTION

The present invention relates to pharmaceutical compositions. Particularly, the present invention relates to oral controlled release (CR) pharmaceutical compositions and processes for their preparation.

### BACKGROUND OF THE INVENTION

Controlled release formulations for administering therapeutically active ingredients are, well known in the art. Solid controlled release formulations are the more common and preferred forms. These solid controlled release formulations can be in tablet form and often comprise:

i) A core of a therapeutically active material which is coated with varying thicknesses of controlled release agents.
ii) Tablet form or granules of controlled release formulations comprise alternating layers of therapeutically active materials and controlled release agent.
iii) Another common form of a controlled release tablet composition comprises matrix system where drug is uniformly dispersed in a polymeric matrix.

The solid controlled release formulations can be in capsule form comprising multiple pellets and or granules with varying degree of thickness or coating of control releasing agent.

Major disadvantages of solid controlled release pharmaceutical compositions are large size of dosage form and weight. The administration of such dosage units present a problem of difficulty in swallowing in children and elder people. Therefore it is desirable to provide such controlled release medicines either in chewable or orally disintegrating solid dosage form or liquid dosage form. In large number of patient group, liquid oral dosage forms are more preferred because it can be swallowed easily without putting additional efforts for chewing which results in better patient compliance.

Liquid controlled release formulations are not as common in the art as solid controlled release formulations. U.S. Pat. No. 2,990,332 discloses a liquid controlled release formulation comprising a suspension of an ion exchange resin in finely divided form complexed with a pharmaceutical active agent. This type of formulation provides a minor delay in release. Further, these formulations are not stable during storage and/or have a short shelf-life due to limited stability. Moreover, these suspensions must be well-mixed prior to administration. Particular problems may arise upon transfer of unmixed suspensions to another container, since a homogeneous sample would not be present in the new container and accurate dosing would not be possible. Another disadvantage of these suspensions is that they are gritty in taste, leaching and cannot give extended and predicted release profile for 12-24 hours. Many of these disadvantages tend to lower patient compliance.

Various other liquid controlled release formulations have been disclosed in the prior art. PCT Application WO1985003000 discloses a sustained release liquid dosage formulation produced by coating a pre-made solid controlled release dosage form with a dual coating and subsequently dispersing the resulting dosage forms in a liquid carrier. Disadvantages with this method include the requirement for a pre-made controlled release form, thus these formulations can not prepared in-situ and they require at least two further processing steps to achieve a liquid dosage form.

US20050095300 discloses a pharmaceutical liquid controlled release suspension comprising NSAID and/or Acetaminophen substantially covered with one layer of controlled release composition dispersed in suspension base made of water miscible cosolvents selected from glycols, alcohols and glycerols. The invention disclosed in US20050095300 use ethyl cellulose and poly methacrylate (pH dependent polymers).

US20080118570 discloses a coated drug/resin complex that comprises a plurality of dry ion exchange resin forms complexed with a drug component to form a drug/resin complex; and a substantially plasticizer-free coating layer having a thickness that comprises a sufficient amount of methacrylate polymer applied at least substantially around a portion of the resin forms to control the release rate of the drug component associated therewith. Ion exchange resins cannot provide extended release profile for 12-24 hrs as it gets dissociated with respect to pH. The methacrylate polymer used for coating is formed from an aqueous dispersion of a neutral copolymer comprising polyethyl acrylate and polymethyl methacrylate.

Although children (0-12 years) comprise approximately 22% of the total population, very few medicines have been specifically tested for them. Often, the adult dose is reduced according to size and weight of the child, which can pose a considerable health risk. Governments around the world are now putting considerable pressure on the pharmaceutical industry to improve this situation by developing more and improved medicines especially for children and elderly.

Conventional dosage forms are administered frequently, as 3-4 times a day. Children and older patients are very vulnerable patient populations for non-compliance of the administration schedule, as the frequent administration of dosage form in these populations is a big challenge.

The pharmaceutical dosage forms should provide the following key advantages for better compliance:
a) Dispensed in a way familiar to the child
b) Easy to swallow
c) Provides fast and long acting action

From a physician's point of view, better delivery systems for children and elderly are also needed because compliance is a major issue for all involved in the healthcare process. The present platform technology can provide the unmet medical needs of improving patient compliance and reducing dosing frequency for pediatric as well as geriatric patients. The pharmaceutical composition to be developed would eventually be in one of the following dosage forms:
1. Suspension: The drug is present in suspension form, as a ready-to-use liquid formulation.
2. Ready for constitution: The drug is present in a powder form to be reconstituted and used within 7-14 days depending on leaching and stability study.
3. Ready for constitution pre-use: The drug is present in a powder or granular form and packed in unit sachet or in a tablet form, to be reconstituted just prior to use.

Accordingly, inspite of existing prior arts still there is a need for improved and stabilized controlled release pharmaceutical composition, preferably liquid, for high solubility and/or medium solubility and/or low solubility drug. The inventors of the present invention have come up with a novel oral controlled release pharmaceutical composition, which is described hereinafter.

### OBJECT OF THE INVENTION

It is an object of the present invention to provide orally ingestible controlled release pharmaceutical compositions.

It is another object of the present invention to provide orally ingestible controlled release pharmaceutical compositions which act for a longer period of time with reduced dosing frequency.

It is another object of the present invention to provide orally ingestible controlled release pharmaceutical compositions with improved stability.

It is another object of the present invention to provide orally ingestible controlled release pharmaceutical compositions with improved geriatric and pediatric patient compliance.

It is still another object of the present invention to provide orally ingestible controlled release pharmaceutical compositions which avoid localized accumulation of the drug leading to local irritation.

It is yet another object of the present invention to provide orally ingestible controlled release pharmaceutical compositions which ensures steady and predictable drug release with minimal inter and intra subject variation.

It is a further object of the present invention to provide orally ingestible controlled release pharmaceutical compositions which delivers more than two incompatible drugs in a single dosage form.

It is a still further object of the present invention to provide orally ingestible controlled release pharmaceutical compositions which delivers more than one active ingredients in multiple doses with different release profiles.

### BRIEF DESCRIPTION OF THE DRAWINGS

FIG. 1 is a graph depicting dissolution profile of the pharmaceutical composition as prepared in example-1.

FIG. 2 is a graph depicting dissolution profile for Ondansetron oral controlled release reconstituted suspension as prepared in example-5.

FIG. 3 is a graph depicting drug release profile for Paracetamol oral SR granules and reconstituted suspension as prepared in example-7.

FIG. 4 is a graph depicting drug release profile for Tamsulosin SR and Finasteride OD tablets as prepared in example-13.

### SUMMARY OF THE INVENTION

The present invention relates to an oral controlled release pharmaceutical composition comprising:
(a) a core selected from the group consisting of ‘drug coated core’ comprising an inert excipient based sphere coated with a drug coating composition and ‘drug loaded core’ comprising at least one drug, a binder and at least one pharmaceutically acceptable excipient;
   wherein the drug is at least one pharmaceutically active ingredient selected from the group consisting of highly soluble drugs, medium soluble drugs and low soluble drugs in an amount of about 0.1% to about 40% of the mass of the composition; and
(b) a polymer dispersion comprising at least one controlled release polymer and at least one pharmaceutically acceptable excipient;
   wherein said pharmaceutical composition has a duration of therapeutic effect for at least about 6 hours to about 30 hours from oral administration.

Typically, the excipient based sphere is microcrystalline cellulose sphere.

Typically, the drug coating composition comprises at least one drug and at least one pharmaceutically acceptable excipient.

In accordance with another embodiment of the present invention said oral controlled release pharmaceutical composition further comprises an in-situ gelling system comprising at least one gelling polymer.

Typically, the highly soluble drug is a drug having a solubility greater than 100 mg/ml in water and which is at least one selected from the group consisting of metformin hydrochloride, propranolol hydrochloride, ranitidine hydrochloride and diltiazem hydrochloride.

Typically, the medium soluble drug is a drug having a solubility in the range of about 1 mg/ml to about 100 mg/ml in water and which is at least one selected from the group consisting of paracetamol, atomoxetine hydrochloride, duloxetine hydrochloride, fluoxetine hydrochloride, paroxetine hydrochloride, tamsulosin hydrochloride, lidocaine and salicylic acid.

Typically, the low soluble drug is a drug having a solubility of less than 1 mg/ml in water and which is at least one selected from the group consisting of ziprasidone, carbamazepine, ondansetron, lornoxicam, diazepam, alprazolam, ketoprofen, naproxen, oxazepam, prednisolone, progesterone and finasteride.

Typically, the controlled release polymer is at least one selected from the group consisting of ethyl cellulose, hydroxylpropyl cellulose, hydroxylpropyl methylcellulose, sodium carboxy methylcellulose, polyacrylates, neutral copolymers based on ethyl acrylate and methylmethacrylate, copolymers of acrylate and methacrylates, polymethacrylates and polyvinyl acetate.

Typically, the amount of said controlled release polymer is in the range of about 5% to about 60% of the mass of the composition.

Typically, the gelling polymer is at least one selected from the group consisting of sodium alginate, calcium alginate, sodium carboxymethyl cellulose, chitosan, xanthan gum and gellan gum.

Typically, the amount of said gelling polymer is in the range of about 5% to about 60% of the mass of the composition.

Typically, the pharmaceutically acceptable excipient is at least one selected from the group consisting of diluents, binders, thickeners, antioxidants, surfactants, glidants, plasticizers, solvents and preservatives.

Typically, said pharmaceutical composition is in a dosage form selected from the group consisting of suspension, tablet, powder, granule and capsule.

Typically, said composition is in the form of granules having particle size in the range of about 150 microns to about 500 microns.

Typically, said composition is stable.

In accordance with another aspect of the present invention there is provided a process for the preparation of an oral controlled release pharmaceutical composition; said process comprising the following steps:
a. preparing a core selected from the group consisting of ‘drug coated core’ comprising an inert excipient based sphere coated with a drug coating composition and ‘drug loaded core’ comprising at least one drug, a binder and at least one pharmaceutically acceptable excipient;
b. preparing a polymer dispersion comprising at least one controlled release polymer and at least one pharmaceutically acceptable excipient;
c. coating the polymer dispersion over the core formed in step (a) to obtain polymer coated granules;
d. optionally preparing an in-situ gelling system comprising at least one gelling polymer and dispersing with said core or polymer dispersion; and
e. converting the obtained granules into pharmaceutically acceptable dosage form.

In accordance with another embodiment of the present invention said process further comprises curing of the granules at a temperature of from about 30° C. to about 80° C. for about 10 minutes to about 48 hours.

### DETAILED DESCRIPTION OF THE INVENTION

The present invention relates to an oral controlled release pharmaceutical composition comprising:
(a) a core selected from the group consisting of ‘drug coated core’ comprising an inert excipient based sphere coated with a drug coating composition and ‘drug loaded core’ comprising at least one drug, a binder and at least one pharmaceutically acceptable excipient;
   wherein the drug is at least one pharmaceutically active ingredient selected from the group consisting of highly soluble drugs, medium soluble drugs and low soluble drugs in an amount of about 0.1% to about 40% of the mass of the composition; and
(b) a polymer dispersion comprising at least one controlled release polymer and at least one pharmaceutically acceptable excipient;
   wherein said pharmaceutical composition has a duration of therapeutic effect for at least about 6 hours to about 30 hours from oral administration.

In one of the embodiments of the present invention the core is ‘drug coated core’ comprising an inert excipient based sphere coated with a drug coating composition. The inert excipient based sphere comprises inert non-pareils which are conventionally used in pharmaceutical industry and are readily available. The inert non-pareils may be of any pharmaceutically acceptable excipient such as starch, sugar, microcrystalline cellulose, vegetable gums, waxes and the like. Preferably, the inert non-pareils are of microcrystalline cellulose. The size of the inert non-pareils may vary from 0.1 mm-2 mm. The sphere may also be prepared by techniques such as granulation or extrusion-spheronization.

Typically, the drug coating composition comprises at least one drug and at least one pharmaceutically acceptable excipient.

In accordance with another embodiment of the present invention the core is ‘drug loaded core’ comprising at least one drug, a binder and at least one pharmaceutically acceptable excipient.

For example, the core may be prepared by mixing one or more pharmaceutically acceptable excipient and the drug, moistening the mixture with water or a solvent, granulating and subsequently drying to obtain granules which may be used as the core. Alternatively, such granules may be compressed into a tablet, which may be used as the core. The core may also be prepared by mixing one or more pharmaceutically acceptable excipient and the drug, wetting with water or organic solvent and mixing in a high shear granulator to form a homogeneous wet mass, extruding the wet mass to form extrudates which are subsequently spheronized to form spheres which may be used as the core.

The controlled release polymer used for preparing polymer dispersion which is in turn used for coating the core is at least one selected from the group consisting of ethyl cellulose, hydroxylpropyl cellulose, hydroxylpropyl methylcellulose, sodium carboxy methylcellulose, polyacrylates, neutral copolymers based on ethyl acrylate and methylmethacrylate, copolymers of acrylate and methacrylates, polymethacrylates and polyvinyl acetate.

Typically, the amount of said controlled release polymer is in the range of about 5% to about 60% of the mass of the composition.

In accordance with another embodiment of the present invention said oral controlled release pharmaceutical composition further comprises an in-situ gelling system comprising at least one gelling polymer.

The gelling polymer used is at least one selected from the group consisting of sodium alginate, calcium alginate, sodium carboxymethyl cellulose, chitosan, xanthan gum and gellan gum. Typically, the amount of said gelling polymer is in the range of about 5% to about 60% of the mass of the composition.

The polymer coated particles are mixed with an in-situ gelling system, which can rapidly form an insoluble gel at acidic pH to control the drug release. The present technology provides two approaches to control the release (1) by pH independent polymer and (1) by gel formed in acidic pH. The pH independent film forming polymer, swells in presence aqueous medium there by controlling the drug release from the core by diffusion process, over a period of 6-30 hrs. The polymer is neutral in nature, so the interaction with ionic (acidic & basic) drugs may not occur. The polymer does not require a plasticizer for film formation and typically, it has a minimum film forming temperature (MFFT) in the range of about 5° C. to 40° C. Additionally, the gel forming polymers form “In-situ gelling systems” which are the systems in liquid form, but transforms into viscous gels in presence of a certain stimuli. The stimuli can be either a change of temperature, pH, presence of particular ions or substrate. The present invention involves the use of gelling polymers like sodium alginate, which forms gel in presence of divalent cations like calcium ions. The cation source is present in the composition itself in the form of calcium carbonate or the like, which has low solubility in neutral pH (formulation pH when reconstituted) but dissolves at acidic pH. When the composition is in contact with the acidic environment such as in presence of gastric fluids the cation source dissolves in presence of an acid and releases cation like calcium, which interacts with the gelling polymer like sodium alginate to form calcium alginate gel.

Typically, the highly soluble drug is a drug having a solubility greater than 100 mg/ml in water and which is at least one selected from the group consisting of metformin hydrochloride, propranolol hydrochloride, ranitidine hydrochloride and diltiazem hydrochloride.

Typically, the medium soluble drug is a drug having a solubility in the range of about 1 mg/ml to about 100 mg/ml in water and which is at least one selected from the group consisting of paracetamol, atomoxetine hydrochloride, duloxetine hydrochloride, fluoxetine hydrochloride, paroxetine hydrochloride, tamsulosin hydrochloride, lidocaine and salicylic acid.

Typically, the low soluble drug is a drug having a solubility of less than 1 mg/ml in water and which is at least one selected from the group consisting of ziprasidone, carbamazepine, ondansetron, lornoxicam, diazepam, alprazolam, ketoprofen, naproxen, oxazepam, prednisolone, progesterone and finasteride.

Typically, said pharmaceutical composition is in a dosage form selected from the group consisting of suspension, tablet, powder, granule and capsule.

Typically, the formulations are in a single dosage form or multiple dosage forms such as when different components are maintained separately and are admixed prior to administration i.e. reconstituted or are sequentially administered or simultaneously co-administered or when two or more of the same dosage form are administered to achieve the required therapeutic dose of drug.

In accordance with one of the embodiments the composition is in the form of orally ingestible micron-sized, polymer coated granules, with a′size in the range of about 150 to 500 microns, preferably not more than 425 microns, more preferably not more than 300 microns.

The finer particles shall help in avoiding grittiness in mouth when taken as suspension. Finer the particles less will be the gravitational force and hence help to prevent settling of particle in a reconstituted suspension. During compression, finer the particles size, less will be the deformation of particles size and hence no cracking of controlled release polymer film, and helps in compressing the granules into tablets.

Typically, the powder form may be in ‘ready for reconstitution’ form.

Alternatively, the powder is in ‘ready for constitution pre-use’ form.

Typically, the suspension is an orally dispersible matrix system.

Alternatively, the suspension may be a multiple unit particulate system.

Typically, the tablet is an orally dispersible matrix system.

Alternatively, the table may be a multiple unit particulate system.

Typically, the powder is an orally dispersible matrix system.

Alternatively, the powder may be a multiple unit particulate system.

The pharmaceutically acceptable excipient used in the preparation of the present composition is at least one selected from the group consisting of diluents, binders, thickeners, antioxidants, surfactants, glidants, plasticizers, solvents and preservatives.

The composition prepared in accordance with the present invention is stable.

### DEFINITIONS

As used in the present specification, the following words and phrases are generally intended to have the meanings as set forth below, except to the extent that the context in which they are used indicates otherwise.

The term “stable” as used herein refers to the stability studies at 40° C./75% RH and 30° C./65% RH for 3 months when there is no change in the dissolution profiles in the present pharmaceutical composition and it complies standard assay limits.

The term “controlled release (CR) polymer” as used herein refers to the pH independent polymers and gelling polymers. Such pH independent polymers include ethyl cellulose, polyacrylates, neutral copolymers based on ethyl acrylate and methylmethacrylate, copolymers of acrylate and methacrylates, polyvinyl acetate or combinations thereof. Such gelling polymers include alginates like sodium alginate, calcium alginate; sodium carboxymethyl cellulose, chitosan, xanthan gum, gellan gum and the like or combinations thereof.

Suspension means a “ready to use” liquid dosage form.

Ready for constitution means a powder formulation that is to be reconstituted before use and which is to be used within 7-14 days depending on leaching and stability study.

Ready for constitution pre-use means a formulation which is either in a powder or granular form packed in unit sachet and it is to be reconstituted just prior to use.

Oral dispersible matrix system means a dosage form that is dispersed in mouth and swallowed with water, convenient for pediatric & geriatric patients.

Multiple unit particulate system means a dosage form that comprises particles having different release profiles that are delivered simultaneously.

MFFT means minimum film forming temperature.

The term “excipients” as used herein means a component of a pharmaceutical product that is not an active ingredient for example, fillers, diluents, diluents, binders, thickeners, antioxidants, surfactants, glidants, plasticizers, preservatives, solvents and the like or combinations thereof. The excipients that are useful in preparing a pharmaceutical composition are generally safe, non-toxic and neither biologically nor otherwise undesirable and are acceptable for pharmaceutical use.

The term “diluent” or “filler” as used herein means inert substances used as fillers to create the desired bulk, flow properties. Such compounds include, by way of example and without limitation, dibasic calcium phosphate, kaolin, sucrose, mannitol, microcrystalline cellulose, powdered cellulose, precipitated calcium carbonate, sorbitol, starch, lactose, glucose and combinations thereof and other such materials known to those of ordinary skill in the art.

The term “binder” as used herein means agents used while making granules of the active ingredient by mixing it with diluent/filler. Such compounds include, by way of example and without limitation, polyvinyl pyrrolidone, pregelatinized starch, starch; hydroxyl propyl methyl cellulose (HPMC), hydroxypropyl cellulose (HPC) and hydroxy ethyl cellulose (HEC) and combinations thereof and other such materials known to those of ordinary skill in the art.

The term “thickener” as used herein means agents used while making insitu gelling system so that when it comes in contact with liquid it swells and thickens or makes the composition viscous. Such compounds include, by way of example and without limitation, sodium alginate, pectin, gellan gum and combinations thereof and other such materials known to those of ordinary skill in the art.

The term “antioxidant” as used herein means an agent, which inhibits oxidation and is thus used to prevent the deterioration of preparations by the oxidative process. Such compounds include, by way of example and without limitation, ascorbic acid, ascorbic palmitate, sodium citrate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisultfite and other such materials known to those of ordinary skill in the art.

The term “preservative” as used herein an agent, which inhibits and/or prevent the deterioration of pharmaceutical preparations. Such compounds include, by way of example and without limitation sodium citrate, sodium benzoate combination thereof and other such materials known to those of ordinary skill in the art.

The term “surfactant” as used herein refers to the substances that help in reducing the surface tension between the two surfaces and helps in dissolving/dispersing the one phase into other. The compounds used as surfactants according to the present invention are selected from the group but are not limited to glycerol monostearate, polysorbates, sodium lauryl sulfate and sucrose esters of fatty acids, polyoxyl hydrogenated castor oil (of various grades), combination thereof and other such materials known to those of ordinary skill in the art.

The term “glidant” as used herein means agents used in formulations to improve flow-properties. Such compounds include, by way of example and without limitation, silica, calcium silicate, magnesium silicate, silicon hydrogel, cornstarch, talc, combinations thereof and other such materials known to those of ordinary skill in the art.

The term “plasticizer” as used herein means additives that increase the plasticity or fluidity of the material to which they are added. Such compounds include but are not limited to triethyl citrate, triacetin, polyethylene glycol-4000, diethyl phthalate, dibutyl phthalate, cetyl alcohol or propylene glycol combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

In accordance with another aspect of the present invention there is provided a process for the preparation of an oral controlled release pharmaceutical composition; said process comprising the following steps:
a. preparing a core selected from the group consisting of ‘drug coated core’ comprising an inert excipient based sphere coated with a drug coating composition and ‘drug loaded core’ comprising at least one drug, a binder and at least one pharmaceutically acceptable excipient;
b. preparing a polymer dispersion comprising at least one controlled release polymer and at least one pharmaceutically acceptable excipient;
c. coating the polymer dispersion over the core formed in step (a) to obtain polymer coated granules;
d. optionally preparing an in-situ gelling system comprising at least one gelling polymer and dispersing with said core or polymer dispersion; and
e. converting the obtained granules into pharmaceutically acceptable dosage form.

In accordance with another embodiment of the present invention said process further comprises curing of the granules at a temperature of from about 30° C. to about 80° C. for about 10 minutes to about 48 hours.

The curing step assures the complete film formation during polymer coating. The curing step is generally a thermal post-treatment which is required to enhance the degree of polymer particle coalescence.

The following examples are provided to enable one skilled in the art to practice the invention and are merely illustrative of the present invention. The examples should not be read as limiting the scope of the present invention.

### EXAMPLES

### Example-1

The present, controlled release pharmaceutical composition can be prepared as shown in table 1 and table 2 and described below.

### Drug Loading Composition

TABLE 1

| Ingredients | mg/unit |
|---|---|
| microcrystalline cellulose spheres | 100.0 |
| (Celphere ® CP 203) | |
| ondansetron | 24.0 |
| hydroxypropyl cellulose (Klucel ® IP) | 6.0 |
| Talc | 10.0 |
| isopropyl alcohol | 130.0 |
| water | 80.0 |
| solid content in coating dispersion | 40.0 |
| weight of ondansetron drug loaded pellets | 140.0 |

### Procedure

Isopropyl alcohol and water were mixed to get a solvent mixture.
Talc and ondansetron were dispersed in ⅔rd of solvent mixture using rotor stator disperser.
Hydroxypropyl cellulose was dissolved in the remaining solvent mixture and mixed with the above dispersion.
Drug dispersion was sprayed on to microcrystalline cellulose spheres to get drug loaded pellets and used for controlled release, coating.

### CR Coating Composition

TABLE 2

| Ingredients | mg/unit |
|---|---|
| Polyvinyl acetate dispersion | 52.5 |
| (Kollicoat ® SR 30 D) | |
| Triethyl citrate | 0.8 |
| Talc | 4.5 |
| Water | 47.2 |
| Solid content in coating dispersion | 21.0 |
| Weight of ondansetron CR coated pellets | 161.0 |

### Procedure

Triethyl citrate was dissolved in half of water and mixed with polyvinyl acetate dispersion.
Talc was dispersed in remaining water using rotor stator disperser and mixed with the above dispersion.
Controlled release coating dispersion was sprayed on to drug loaded pellets to get CR coated granules.
The CR coated granules were cured for 4 hours at 55° C.

### Ondansetron Granules for Ingestion Composition

CR coated granules were mixed with extra granular composition as below.
Extra granular composition

TABLE 3

| Ingredients | mg/unit |
|---|---|
| Ondansetron CR coated granules | 163.0 |
| Sugar | 2137.0 |
| Microcrystalline cellulose and Sodium | 100.0 |
| carboxymethyl cellulose (Avicel ® CL611) | |

The CR granules were mixed with sugar (milled through 0.5 mm sieve) and Avicel® CL 611 and filled into amber glass bottles.

### Example-2

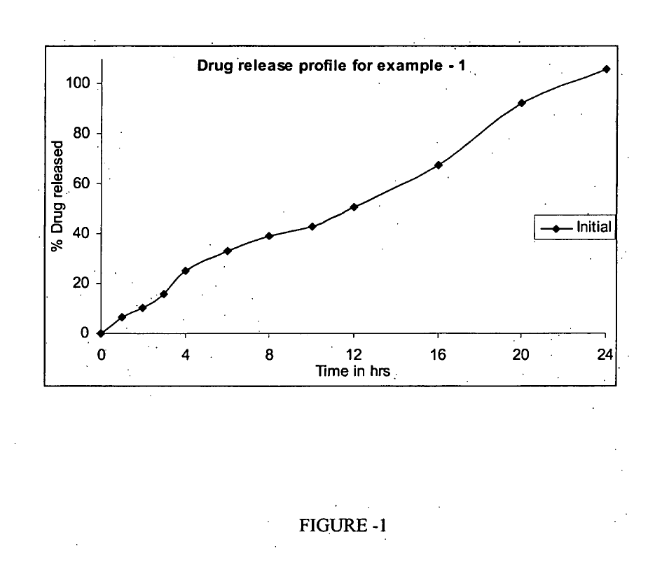

The pharmaceutical composition as prepared in example-1 was studied for dissolution and the results obtained are shown in table 4 and FIG. 1.

### Dissolution Conditions—

USP dissolution apparatus I (Basket), 0.01N HCl, 37° C. ±0.5° C., 75 rpm

TABLE 4

| | % dissolution |
|---|---|
| Time in hours | Test example -1 |
| 1 | 6.4 |
| 2 | 10.3 |
| 3 | 15.9 |
| 4 | 25.1 |
| 6 | 33.3 |
| 8 | 39.2 |
| 10 | 42.8 |
| 12 | 50.6 |
| 16 | 67.6 |
| 20 | 92.5 |
| 24 | 105.6 |

### Example-3

### Stability Studies:

The controlled release pharmaceutical composition as prepared in example-1 was kept for stability studies at 40° C./75% RH and 30° C./65% RH for 3 months and the results are shown in table 5.

TABLE 5

| | | Test | | |
|---|---|---|---|---|
| | | | 40° C./ | 30° C./ |
| | | | 75% RH | 65% RH |
| | | Initial | 2M | 3M |
| % Assay - (limit 90-110) | | 102.2 | 103.5 | 100.9 |
| % Loss on drying | | 0.82 | 0.93 | 0.75 |
| % Dissolution (N = 6) | 1 h | 6.4 | 3.4 | 4.0 |
| in 0.01N HCl, USP | 2 h | 10.3 | 12.6 | 11.2 |
| Apparatus I, 100 rpm | 3 h | 15.9 | 21.4 | 19.0 |
| | 4 h | 25.1 | 29.0 | 25.0 |
| | 6 h | 33.3 | 41.6 | 33.1 |
| | 8 h | 39.2 | 52.3 | 39.1 |
| | 10 h | 42.8 | 60.1 | 44.2 |
| | 12 h | 50.6 | 65.8 | 52.5 |
| | 16 h | 67.6 | 73.7 | 60.3 |
| | 20 h | 92.5 | 79.4 | 81.1 |
| | 24 h | 105.6 | 83.6 | 99.3 |

### Example-4

The controlled release pharmaceutical composition as described in example-1-CR coating composition can also be converted into pharmaceutical dosage forms as shown in table 6, table 7 and table 8 and described below.

### 1. Ondansetron Granules for Ingestion Composition

CR coated granules were mixed with extra granular composition as below.
Extra granular composition

TABLE 6

| Ingredients | mg/unit |
|---|---|
| Ondansetron CR coated granules | 163.0 |
| Sugar | 2137.0 |
| Microcrystalline Cellulose and Sodium | 100.0 |
| Carboxymethyl Cellulose (Avicel ® CL611) | |

The CR granules were mixed with Sugar (milled through 0.5 mm sieve) and Avicel® CL 611 and filled into amber glass bottles.

### 2. Ondansetron Oral Controlled Release Powder for Suspension

Protective coating of Ondansetron controlled release

CR coated pellets were further coated with amino methacrylate copolymer (Eudragit® EPO) as per composition below.
Composition for Protective coating

TABLE 7

| Ingredients | mg/unit |
|---|---|
| Amino Methacrylate Copolymer | 28.0 |
| (Eudragit ® EPO) | |
| Sodium Lauryl Sulfate | 2.8 |
| Stearic Acid | 4.2 |
| Talc | 14.0 |
| Water | 277.0 |
| Solid content in coating dispersion | 49.0 |
| Weight of Ondansetron CR coated pellets | 212.0 |

### Procedure

Eudragit® EPO, Sodium lauryl sulfate and stearic acid were dispersed successively into the water, using a high shear (rotor stator) mixer, until no polymer remains on the surface.
Talc was added and dispersed with a high shear mixer (rotor stator) for 30 minutes. A colloidal solution was formed which appears white due to the talc added.
Eudragit® EPO coating dispersion was sprayed on to Ondansetron CR coated pellets of example-1 in Fluid Bed Processor to obtain Ondansetron Eudragit® EPO coated granules.
Composition for Ondansetron oral controlled release powder for suspension

TABLE 8

| Ingredients | mg/unit |
|---|---|
| Ondansetron Eudragit ® EPO coated granules | 212.0 |
| Sugar | 2088.0 |
| Microcrystalline Cellulose and Sodium | 150.0 |
| Carboxymethyl Cellulose (Avicel ® CL611) | |
| Total weight of powder for suspension | 2450.0 |

### Procedure

The Ondansetron Eudragit® EPO coated granules were mixed with sugar (milled through 0.5 mm sieve) and Avicel® CL 611 to get powder for suspension.
Above powder for suspension equivalent to 12 units (29.4 g) was filled into glass bottles marked to 60 ml.
The powder was reconstituted with water up to 60 ml mark to get reconstituted suspension.

### Example-5

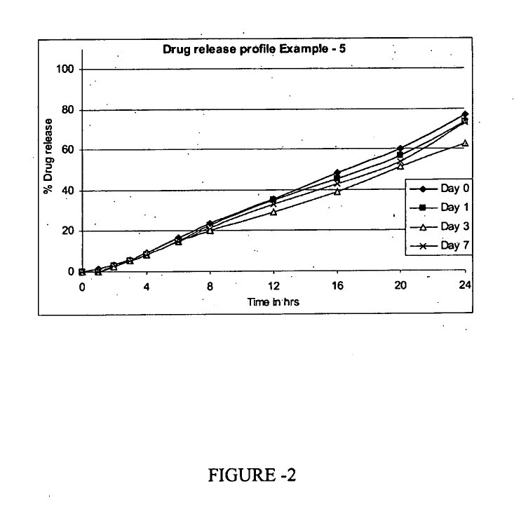

Ondansetron oral controlled release powder for suspension as prepared in example-4 was studied for drug release profile after reconstitution and the results are shown in table 9 and FIG. 2.
Drug release study was carried out at 0, 1, 3 & 7 days on each unit of 5 ml of reconstituted suspension.

### Dissolution Conditions—

USP dissolution apparatus I (Basket), 0.01N HCl, 37° C. ±0.5° C., 75 rpm.

TABLE 9
% Dissolution

| Time in | | | | |
|---|---|---|---|---|
| hours | Day 0 | Day 1 | Day 3 | Day 7 |
| 0 | 0.0 | 0.0 | 0.0 | 0.0 |
| 1 | 1.6 | 0.0 | 0.0 | 0.0 |
| 2 | 3.3 | 2.8 | 2.7 | 2.8 |
| 3 | 6.0 | 5.2 | 5.4 | 5.7 |
| 4 | 9.5 | 8.2 | 8.6 | 8.6 |
| 6 | 16.7 | 15.3 | 14.7 | 15.0 |
| 8 | 23.6 | 22.5 | 20.3 | 21.6 |
| 12 | 35.4 | 34.9 | 29.3 | 33.2 |
| 16 | 48.2 | 45.5 | 38.8 | 42.7 |
| 20 | 60.4 | 56.5 | 51.1 | 54.0 |
| 24 | 76.8 | 73.6 | 62.8 | 72.9 |

### Example-6

The present controlled release pharmaceutical composition can also be prepared as shown in tables 10, 11 and 12 and described below.
Oral controlled release suspension

### Core Granules Composition

TABLE 10

| Ingredients | mg/unit |
|---|---|
| Ondansetron | 24.00 |
| Dibasic calcium phosphate dihydrate | 7.93 |
| Colloidal silicon dioxide | 0.20 |
| Povidone K 30 | 5.76 |
| Weight of granules | 37.89 |

### Procedure

Povidone was dissolved in, sufficient water using mechanical stirrer to get binder solution.
Dibasic calcium phosphate dihydrate, ondansetron & colloidal silicon dioxide were mixed and loaded into bowl of a fluid bed processor with top spray assembly.
The binder was sprayed on to powder to get the granules and the granules were dried in fluid bed processor.
The granules fraction was passed through sieve #80 and retained on sieve #100 & used for CR coating.

### CR Coating Composition

TABLE 11

| Ingredients | mg/unit |
|---|---|
| Ethyl Acrylate and Methyl Methacrylate | 30.77 |
| Copolymer Dispersion (Eudragit ® NE 30D) | |
| Talc | 4.615 |
| Water | 33.85 |
| Weight of coating | 13.85 |

### Procedure

Talc was dispersed in water and mixed with Eudragit® NE 30D and the dispersion was sprayed on the granules in a fluid bed processor with bottom spray assembly.
The granules were cured with 0.5% w/w of colloidal silicon dioxide at 45° C. for 24 hrs to get CR granules.

### Extra Granular Composition

TABLE 12

| Ingredients | mg/unit |
|---|---|
| Sugar | 6000 |
| Sodium Alginate | 150 |
| Sodium Citrate | 10 |
| Calcium Carbonate | 40 |
| Weight of extra granular ingredients | 6200 |

### Procedure

CR granules were mixed with extragranular composition containing sugar, sodium alginate, sodium citrate & calcium carbonate to get ondansetron CR powder for suspension.
Ondansetron CR Powder for Suspension equivalent to 24 mg of Ondansetron was dispersed in water to get 10 ml suspension.

### Example-7

The present controlled release pharmaceutical composition can also be prepared as shown in tables 13, 14 and 15 and described below.
Paracetamol oral controlled release suspension

### Core Granules Composition

TABLE 13

| Ingredients | mg/unit |
|---|---|
| Paracetamol | 250 |
| Dicalcium Phosphate Dihydrate | 130 |
| Povidone K 30 | 20 |
| Total Granules | 400 |

### Procedure

Povidone was dissolved in sufficient water using mechanical stirrer to get binder solution.
Dibasic calcium phosphate dihydrate, paracetamol & colloidal silicon dioxide were mixed and loaded into bowl of a fluid bed processor with top, spray assembly.
The binder was sprayed on to powder to get the granules and the granules were dried in fluid bed processor.
The granules fraction was passed through sieve #80 and retained on sieve #100 & used for SR coating.

### SR Coating Composition

TABLE 14

| Ingredients | mg/unit |
|---|---|
| Ethyl Acrylate and Methyl Methacrylate | 200 |
| Copolymer Dispersion (Eudragit NE 30 D) | |
| Talc | 40 |
| Water | 260 |
| Paracetamol SR coated granules | 500 |

### Procedure

Talc was dispersed in water and mixed with Eudragit® NE 30D and the dispersion was sprayed on the granules in a fluid bed processor with bottom spray assembly.
The granules were cured with 0.5 w/w of colloidal silicon dioxide at 45° C. for 24 hrs to get SR coated granules.

### Extra Granular Composition

TABLE 15

| Ingredients | mg/unit |
|---|---|
| Sodium Alginate | 150 |
| Sodium Citrate | 10 |
| Calcium Carbonate | 40 |
| Sugar | 6000 |
| Weight of extra granular ingredients | 6200 |

### Procedure

SR coated granules were mixed with extragranular composition containing sugar,
sodium alginate, sodium citrate & calcium carbonate to get paracetamol SR powder for suspension.
Paracetamol SR powder for suspension equivalent to 250 mg of paracetamol was dispersed in water to get 10 ml suspension.

### Example-8

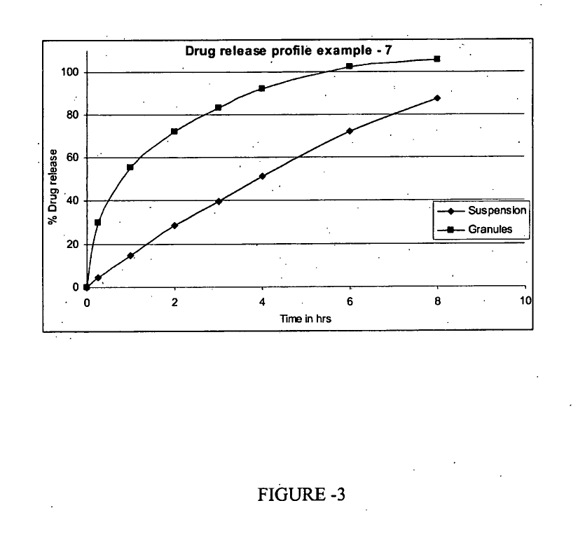

The pharmaceutical composition as prepared in example-7 was studied for dissolution for both SR coated granules and reconstituted suspension the results obtained are shown in table 16 and FIG. 3.

Dissolution condition: 900 mL Acetate buffer pH 4.5, Apparatus USP type II, 100 rpm

TABLE 16
% Dissolution of example - 7

| Time (Hrs) | Suspension | Granules |
|---|---|---|
| 0.25 | 4.4 | 29.9 |
| 1.0 | 14.6 | 55.4 |
| 2.0 | 28.7 | 72.2 |
| 3.0 | 39.7 | 83.4 |
| 4.0 | 51.3 | 91.9 |
| 6.0 | 72.1 | 102.0 |
| 8.0 | 87.4 | 105.3 |

### Example-9

The present controlled release pharmaceutical composition can also be prepared as oral dispersible tablets containing SR lornoxicam as shown in tables 17, 18 and 19 and described below.

### Lornoxicam SR OD Tablets

### Drug Loading Composition

TABLE 17

| Ingredients | mg/unit |
|---|---|
| Microcrystalline Cellulose Spheres | 60.0 |
| (Celphere ® CP 203) | |
| Lornoxicam | 16.0 |
| Hydroxypropyl cellulose | 3.0 |
| Talc | 5.0 |
| Isopropyl alcohol | 86.0 |
| Water | 50.0 |
| Solid content in dispersion | 24.0 |
| Weight of drug loaded pellets | 84.0 |

### Procedure

Isopropyl alcohol & water were mixed to get solvent mixture.
Talc and lornoxicam were dispersed in ⅔rd of solvent mixture using rotor stator disperser.
Hydroxypropyl cellulose was dissolved in remaining solvent mixture and mixed with above dispersion.
Drug dispersion was sprayed on to microcrystalline cellulose spheres to get drug loaded pellets and used for sustained release coating.

### SR Coating Composition

TABLE 18

| Ingredients | mg/unit |
|---|---|
| Polyvinyl Acetate Dispersion | 22.0 |
| (Kollicoat ® SR 30 D) | |
| Kollicoat IR ® | 2.8 |
| Triethyl Citrate | 0.5 |
| Talc | 2.7 |
| Water | 35.0 |
| Solid content in dispersion | 12.6 |
| Weight of Lornoxicam SR coated pellets | 96.6 |

### Procedure

Triethyl citrate was dissolved in half of water and mixed with polyvinyl acetate dispersion.
Talc was dispersed in remaining water using rotor stator disperser and mixed with the above dispersion.
Sustained release coating dispersion was sprayed on to drug loaded pellets to get SR coated pellets.
The SR coated pellets were cured for 20 minutes at inlet of 55° C. in fluid bed coater.

### Composition for SR Oral Dispersible Tablet

TABLE 19

| Ingredients | mg/unit |
|---|---|
| Lornoxicam SR coated pellets | 96.6 |
| F - Melt ® | 150.0 |
| Crosspovidone | 20.0 |
| Mannitol | 131.4 |
| Magnesium Stearate | 2.0 |
| Tablet Weight | 400.0 |

### Procedure

F-Melt®, crospovidone, mannitol and lornoxicam SR coated pellets were passed through sieve #30, lubricated with magnesium stearate.
The blend was compressed into tablets using 10 mm standard concave punches.
The tablets were tested for disintegration time in water, and was found 20 seconds

### Example-10

The present controlled release pharmaceutical composition can also be prepared as sustained release capsules as shown in tables 20 and 21 and described below.

### Lomoxicam SR Capsules

### Drug Loading Composition

TABLE 20

| Ingredients | mg/unit |
|---|---|
| Microcrystalline Cellulose Spheres | 60.0 |
| (Celphere ® CP 203) | |
| Lornoxicam | 16.0 |
| Hydroxypropyl Cellulose | 3.0 |
| Talc | 5.0 |
| Isopropyl Alcohol | 86.0 |
| Water | 50.0 |
| Solid content in dispersion | 24.0 |
| Weight of drug loaded pellets | 84.0 |

### Procedure

Isopropyl alcohol & water were mixed to get solvent mixture.
Talc and lornoxicam were dispersed in ⅔rd of solvent mixture using rotor stator disperser.
Hydroxypropyl cellulose was dissolved in remaining solvent mixture and mixed with above dispersion.
Drug dispersion was sprayed on to microcrystalline cellulose spheres to get drug loaded pellets and used for sustained release coating.

### SR Coating Composition

TABLE 21

| Ingredients | mg/unit |
|---|---|
| Polyvinyl Acetate Dispersion | 22.0 |
| (Kollicoat ® SR 30 D) | |
| Kollicoat IR ® | 2.8 |
| Triethyl Citrate | 0.5 |
| Talc | 2.7 |
| Water | 35.0 |
| Solid content in dispersion | 12.6 |
| Weight of Lornoxicam SR coated pellets | 96.6 |

### Procedure

Triethyl citrate was dissolved in half of water and mixed with polyvinyl acetate dispersion.
Talc was dispersed in remaining water using rotor stator disperser and mixed with the above dispersion.
Sustained release coating dispersion was sprayed on to drug loaded pellets to get SR coated pellets.
The SR coated pellets were cured for 20 minutes at inlet of 55° C. in fluid bed coater.
Lornoxicam SR coated pellets were filled into capsules to get lornoxicam SR capsules.

### Example-11

The present controlled release pharmaceutical composition can be prepared as shown in tables 22, 23 and 24 and described below.

### Propranolol Oral SR Granules

### Drug Loading Composition

TABLE 22

| Ingredients | mg/unit |
|---|---|
| Microcrystalline Cellulose Spheres | 100.0 |
| (Celphere ® CP 203) | |
| Propranolol Hydrochloride | 40.0 |
| Hydroxypropyl Cellulose | 4.0 |
| Talc | 6.0 |
| Water | 600.0 |
| Solid content in dispersion | 50.0 |
| Weight of drug loaded pellets | 150.0 |

### Procedure

Propranolol hydrochloride was dissolved in ¾^{th} of water.
Talc and hydroxypropyl cellulose were dispersed in remaining water and mixed with above dispersion.
Drug dispersion was sprayed on to microcrystalline cellulose spheres to get drug loaded pellets and used for sustained release coating.

### SR Coating Composition

TABLE 23

| Ingredients | mg/unit |
|---|---|
| Polyvinyl Acetate Dispersion | 120.0 |
| (Kollicoat ® SR 30 D) | |
| Triethyl Citrate | 2.0 |
| Talc | 12.0 |
| Water | 116.0 |
| Solid content in coating dispersion | 50.0 |
| Weight of Propranolol SR coated pellets | 200.0 |

### Procedure

Triethyl citrate was dissolved in half of water and mixed with polyvinyl acetate dispersion.
Talc was dispersed in remaining water using rotor stator disperser and mixed with the above dispersion.
Sustained release coating dispersion was sprayed on to drug loaded pellets to get SR coated pellets.

### Propranolol Granules for Ingestion Composition

SR coated pellets were mixed with extra granular composition as below.
Extra granular composition

TABLE 24

| Ingredients | mg/unit |
|---|---|
| Propranolol SR coated pellets | 200.0 |
| Sugar | 2150.0 |
| Microcrystalline Cellulose and Sodium | 150.0 |
| Carboxymethyl Cellulose (Avicel ® CL611) | |
| Total weight of powder for suspension | 2500.0 |

The SR coated pellets were mixed with Sugar (milled through 0.5 mm sieve) and Avicel® CL 611 and filled into amber glass bottles.

### Example-12

The present controlled release pharmaceutical composition can be prepared as shown in tables 25, 26 and 27 and described below.

### Atomoxetine Oral SR Granules

### Drug Loading Composition

TABLE 25

| Ingredients | mg/unit |
|---|---|
| Microcrystalline Cellulose Spheres | 100.0 |
| (Celphere ® CP 203) | |
| Atomoxetine Hydrochloride | 40.0 |
| Hydroxypropyl Cellulose | 4.0 |
| Talc | 6.0 |
| Water | 750.0 |
| Solid content in dispersion | 50.0 |
| Weight of drug loaded pellets | 150.0 |

### Procedure

Atomoxetine hydrochloride was dispersed in ⅘^{th} of water.
Talc and hydroxypropyl cellulose were, dispersed in remaining water and mixed with above dispersion.
Drug dispersion was sprayed on to microcrystalline cellulose spheres to get drug loaded pellets and used for sustained release coating.

### SR Coating Composition

TABLE 26

| Ingredients | mg/unit |
|---|---|
| Polyvinyl Acetate Dispersion | 120.0 |
| (Kollicoat ® SR 30 D) | |
| Triethyl Citrate | 2.0 |
| Talc | 12.0 |
| Water | 116.0 |
| Solid content in coating dispersion | 50.0 |
| Weight of Atomoxetine SR coated pellets | 200.0 |

### Procedure

Triethyl citrate was dissolved in half of water and mixed with polyvinyl acetate dispersion.
Talc was dispersed in remaining water using rotor stator disperser and mixed with the above dispersion.
Sustained release coating dispersion was sprayed on to drug loaded pellets to get SR coated pellets.

### Atomoxetine Granules for Ingestion Composition

SR coated granules were mixed with extra granular composition as below.
Extra granular composition

TABLE 27

| Ingredients | mg/unit |
|---|---|
| Atomoxetine SR coated granules | 200.0 |
| Sugar | 2150.0 |
| Microcrystalline Cellulose and Sodium | 150.0 |
| Carboxymethyl Cellulose (Avicel ® CL611) | |
| Total weight of powder for suspension | 2500.0 |

The SR pellets mixed with Sugar (milled through 0.5 mm sieve) and Avicel® CL 611 and filled into amber glass bottles.

### Example-13

The present controlled release pharmaceutical composition can also be prepared as oral dispersible tablet containing tamsulosin SR and finasteride as shown in tables 28, 29, 30 and 31 and described below.

### Tamsulosin SR+Finasteride OD Tablets

### Drug Loading Composition

TABLE 28

| Ingredients | mg/unit |
|---|---|
| Microcrystalline Cellulose Spheres | 50.0 |
| (Celphere ® CP 203) | |
| Tamsulosin Hydrochloride | 0.8 |
| Hydroxypropyl Cellulose | 3.0 |
| Talc | 1.2 |
| Water | 95.0 |
| Solid content in dispersion | 5.0 |
| Weight of drug loaded pellets | 55.0 |

### Procedure

Tamsulosin hydrochloride was dissolved in ¾^{th} water.
Talc and hydroxypropyl cellulose was dissolved in remaining water and mixed with above solution.
Drug dispersion was sprayed on to microcrystalline cellulose spheres to get drug loaded pellets and used for sustained release coating.

### SR Coating Composition

TABLE 29

| Ingredients | mg/unit |
|---|---|
| Polyvinyl Acetate Dispersion | 12.0 |
| (Kollicoat ® SR 30 D) | |
| Triethyl Citrate | 0.2 |
| Talc | 1.2 |
| Water | 11.6 |
| Solid content in coating dispersion | 5.0 |
| Weight of Tamsulosin SR coated pellets | 60.0 |

### Procedure

Triethyl citrate was dissolved in half of water and mixed with polyvinyl acetate dispersion.
Talc was dispersed in remaining water using rotor stator disperser and mixed with the above dispersion.
Sustained release coating dispersion was sprayed on to drug loaded pellets to get tamsulosin SR coated pellets

### Drug Loading Composition

TABLE 30

| Ingredients | mg/unit |
|---|---|
| Finasteride | 5.0 |
| Hydroxypropyl Cellulose | 3.0 |
| Talc | 2.0 |
| Alcohol | 60.0 |
| Water | 30.0 |
| Solid content in dispersion | 10.0 |
| Weight of Tamsulosin SR and Finasteride | 65.0 |
| pellets | |

### Procedure

Finasteride was dissolved in alcohol.
Hydroxypropyl cellulose was dissolved in half the quantity of water and mixed with above solution.
Talc was dispersed in remaining water using rotor stator disperser and mixed with above dispersion.
Drug dispersion was sprayed on to tamsulosin SR coated pellets to get tamsulosin SR and finasteride pellets.

### Composition for Oral Dispersible Tamsulosin SR and Finasteride OD Tablet

TABLE 31

| Ingredients | mg/unit |
|---|---|
| Tamsulosin SR and Finasteride pellets | 65.0 |
| F - Melt ® | 150.0 |
| Crosspovidone | 15.0 |
| Mannitol | 118.0 |
| Magnesium Stearate | 2.0 |
| Tablet Weight | 350.0 |

### Procedure

F-Melt®, crospovidone, mannitol and tamsulosin SR and finasteride pellets were passed through sieve #30, lubricated with magnesium stearate.
The blend was compressed into tablets using 10 mm standard concave punches.

### Example-14

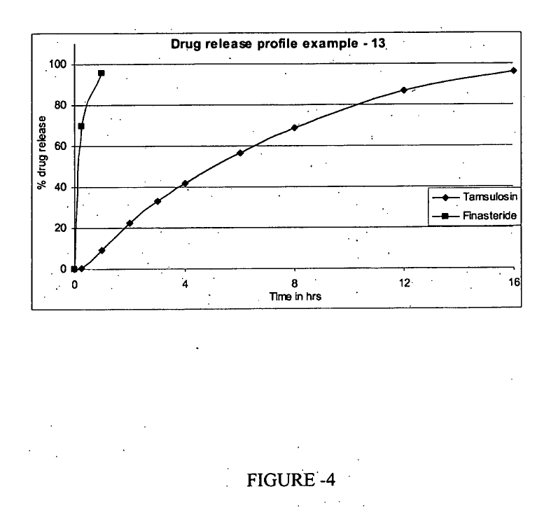

The pharmaceutical composition as prepared in example-13 was studied for dissolution for tamsulosin SR and finasteride OD tablets the results obtained are shown in table 32 and FIG. 4.

Dissolution condition: 900 mL Phosphate buffer pH 6.8, Apparatus USP type II, 75 rpm

TABLE 32
% Dissolution of example - 13

| Time (Hrs) | Tamsulosin | Finasteride |
|---|---|---|
| 0.25 | 0.5 | 69.6 |
| 1 | 9.4 | 95.2 |
| 2 | 22.2 | |
| 3 | 32.9 | |
| 4 | 41.8 | |
| 6 | 56.3 | |
| 8 | 68.8 | |
| 12 | 86.7 | |
| 16 | 95.8 | |

While considerable emphasis has been placed herein on the specific ingredients of the preferred composition, it will be appreciated that many additional ingredients can be added and that many changes can be made in the preferred composition without departing from the principles of the invention. These and other changes in the preferred composition of the invention will be apparent to those skilled in the art from the disclosure herein, whereby it is to be distinctly understood that the foregoing descriptive matter is to be interpreted merely as illustrative of the invention and not as a limitation.

1. An oral controlled release pharmaceutical composition comprising:
(a) a core selected from the group consisting of ‘drug coated core’ comprising an inert excipient based sphere coated with a drug coating composition and ‘drug loaded core’ comprising at least one drug, a binder and at least one pharmaceutically acceptable excipient;
wherein the drug is at least one pharmaceutically active ingredient selected from the group consisting of highly soluble drugs, medium soluble drugs and low soluble drugs in an amount of about 0.1% to about 40% of the mass of the composition; and
(b) a polymer dispersion comprising at least one controlled release polymer and at least one pharmaceutically acceptable excipient;
wherein said pharmaceutical composition has a duration of therapeutic effect for at least about 6 hours to about 30 hours from oral administration.

2. The oral, controlled release pharmaceutical composition as claimed in claim 1, wherein the excipient based sphere is microcrystalline cellulose sphere.

3. The oral controlled release pharmaceutical composition as claimed in claim 1, wherein the drug coating composition comprises at least one drug and at least one pharmaceutically acceptable excipient.

4. The oral controlled release pharmaceutical composition as claimed in claim 1, further comprises an in-situ gelling system comprising at least one gelling polymer.

5. The oral controlled release pharmaceutical composition as claimed in claim 1, wherein the highly soluble drug is a drug having a solubility greater than 100 mg/ml in water and which is at least one selected from the group consisting of metformin hydrochloride, propranolol hydrochloride, ranitidine hydrochloride and diltiazem hydrochloride.

6. The oral controlled release pharmaceutical composition as claimed in claim 1, wherein the medium soluble drug is a drug having a solubility in the range of about 1 mg/ml to about 100 mg/ml in water and which is at least one selected from the group consisting of paracetamol, atomoxetine hydrochloride, duloxetine hydrochloride, fluoxetine hydrochloride, paroxetine hydrochloride, tamsulosin hydrochloride, lidocaine and salicylic acid.

7. The oral controlled release pharmaceutical composition as claimed in claim 1, wherein the low soluble drug is a drug having a solubility of less than 1 mg/ml in water and which is at least one selected from the group consisting of ziprasidone, carbamazepine, ondansetron, lornoxicam, diazepam, alprazolam, ketoprofen, naproxen, oxazepam, prednisolone, progesterone and finasteride.

8. The oral controlled release pharmaceutical composition as claimed in claim 1, wherein said controlled release polymer is at least one selected from the group consisting of ethyl cellulose, hydroxylpropyl cellulose, hydroxylpropyl methylcellulose, sodium carboxy methylcellulose, polyacrylates, neutral copolymers based on ethyl acrylate and methylmethacrylate, copolymers, of acrylate and methacrylates, polymethacrylates and polyvinyl acetate.

9. The oral controlled release pharmaceutical composition as claimed in claim 1, wherein the amount of said controlled release polymer is in the range of about 5% to about 60% of the mass of the composition.

10. The oral controlled release pharmaceutical composition as claimed in claim 4, wherein said gelling polymer is at least one selected from the group consisting of sodium alginate, calcium alginate, sodium carboxymethyl cellulose, chitosan, xanthan gum and gellan gum.

11. The oral controlled release pharmaceutical composition as claimed in claim 4, wherein the amount of said gelling polymer is in the range of about 5% to about 60% of the mass of the composition.

12. The oral controlled release pharmaceutical composition as claimed in claim 1, wherein said pharmaceutically acceptable excipient is at least one selected from the group consisting of diluents, binders, thickeners, antioxidants, surfactants, glidants, plasticizers, solvents and preservatives.

13. The oral controlled release pharmaceutical composition as claimed in claim 1, wherein said pharmaceutical composition is in a dosage form selected from the group consisting of suspension, tablet, powder, granule and capsule.

14. The oral controlled release pharmaceutical composition as claimed in claim 1, wherein said composition is in the form of granules having particle size in the range of about 150 microns to about 500 microns.

15. The oral controlled release pharmaceutical composition as claimed in claim 1, wherein said composition is stable.

16. A process for the preparation of an oral controlled release pharmaceutical composition; said process comprising the following steps:
(a) preparing a core selected from the group consisting of ‘drug coated core’ comprising an inert excipient based sphere coated with a drug coating composition and ‘drug loaded core’ comprising at least one drug, a binder and at least one pharmaceutically acceptable excipient;
(b) preparing a polymer dispersion comprising at least one controlled release polymer and at least one pharmaceutically acceptable excipient;
(c) coating the polymer dispersion over the core formed in step (a) to obtain polymer coated granules;
(d) optionally preparing an in-situ gelling system comprising at least one gelling polymer and dispersing with said core or polymer dispersion; and
(e) converting the obtained granules into pharmaceutically acceptable dosage form.

17. The process as claimed in claim 16, further comprises curing of the granules at a temperature of about 30° C. to about 80° C. for about 10 minutes to about 48 hours.
